(19) United States
(12) Patent Application Publication
(10) Pub. No.: US 2010/0130877 A1
(43) Pub. Date: May 27, 2010

(54) BODY COMPRESSOR AND BLOOD PRESSURE MONITOR

(75) Inventors: Kenichi Matsumoto, Nishitokyo-shi (JP); Takashi Nakanishi, Nishitokyo-shi (JP)

Correspondence Address: KANESAKA BERNER AND PARTNERS LLP, 1700 DIAGONAL RD, SUITE 310, ALEXANDRIA, VA, 22314-2848, US

(73) Assignee: CITIZEN HOLDINGS CO., LTD., Nishitokyo-shi (JP)

(21) Appl. No.: 12/450,397

(22) PCT Filed: Mar. 24, 2008
(86) PCT No.: PCT/JP2008/055357

§ 371 Date: Sep. 24, 2009

(30) Foreign Application Priority Data
Mar. 30, 2007 (JP) 2007-093015

(51) Int. Cl. A61B 5/022; A61B 17/135

(52) U.S. Cl. 600/499; 606/202

(57) ABSTRACT

It is an object of the present invention to provide a body compressor and a blood pressure monitor, wherein an overinsertion preventing member is provided to prevent the other end of a band from being excessively inserted in a metal clip when the body compressor is taken off from the upper arm and stored, in order to enable the improvement of operability and the prevention of fluffing of a front cover in the vicinity of one end due to friction.

A body compressor 1 includes a band 10, a metal clip 14, a hook-and-loop fastener 2 and an overinsertion preventing member 5. The overinsertion preventing member 5 which comes into contact with the metal clip 14 is provided at a position a predetermined distance apart from one end of the band 10 toward the other end thereof.

### TECHNICAL FIELD

The present invention relates to a body compressor and a blood pressure monitor. More particularly, the present invention relates to a body compressor which is used in an automatic blood pressure monitor and which prevents the diameter of a band from being excessively reduced during storage so that, for example, operability can be improved. The present invention also relates to a blood pressure monitor using this body compressor.

### BACKGROUND ART

Recently, blood pressure monitors have come into wide use not only in, for example, hospitals but also in general households for the purpose of health care. The blood pressure monitors for the general households are typically automatic blood pressure monitors that are easy to operate.

Automatic blood pressure monitors based on various measurement methods and configurations have been developed, and an oscillometric electronic blood pressure monitor is one such blood pressure monitor. This oscillometric electronic blood pressure monitor detects a pulse wave superposed on the inner pressure of an air bag, and calculates a blood pressure on the basis of changes in the amplitude of the pulse wave. This oscillometric electronic blood pressure monitor includes a body compressor, a pressure sensor, a pump, an exhaust valve, an information processing unit, an operation switch and a display. The body compressor (generally called a cuff) is wound around the upper arm, and has an air bag. The pressure sensor, the pump and the exhaust valve are in communication with the air bag through a tube. The information processing unit is connected to the pressure sensor, the pump and the exhaust valve. The operation switch and the display are connected to the information processing unit.

Body compressors of various configurations are used in the automatic blood pressure monitors.

One such body compressor includes a band, an annular metal clip and a hook-and-loop fastener. The band has an air bag, a front cover and a rear cover. The air bag is interposed between the front cover and the rear cover. The annular metal clip is attached to longitudinal one end of the band. The hook-and-loop fastener is inserted into the metal clip, and fixes the other end of the band folded via the metal clip. This body compressor is wound around the upper arm of a user. This body compressor has a simple configuration, and is therefore easy to operate and sold at a low price. Owing to these advantages, this body compressor enjoys longstanding popularity.

Moreover, various technical improvements have been made in the body compressor to, for example, improve ease of use.

(Related Art Examples)

For example, Patent document 1 discloses the technique for a blood pressure measurement cuff (body compressor) substantially similar in configuration to the body compressor described above. In this blood pressure measurement cuff, an anti-drop expansion is formed at the other end of the band inserted in a metal clip to prevent the other end of the band from dropping from the metal clip.

According to this technique, troublesome work can be eliminated. The troublesome work means the insertion of the other end of the band into the annular metal clip whenever a user winds the body compressor around the upper arm. Patent document 1: Japanese Utility Model Publication Laid-open No. 48-20282

### DISCLOSURE OF THE INVENTION

### Problems to be solved by the invention

However, in the case of the body compressor described in Patent document 1, the other end of the band may be excessively inserted in the metal clip when the user is to take off and store the body compressor after a blood pressure measurement. In such a case, the user has to expand, to a diameter at which the upper arm can be inserted, the body compressor which has been diametrically reduced by the excessive insertion. This expanding work is troublesome work. Therefore, there is a problem that operability disadvantageously decreases.

In general, the body compressor described above includes a hook-and-loop fastener. When stored, this body compressor is reduced into a predetermined diameter, and then its other end is folded via the metal clip. Further, a hook surface at the other end is joined to a loop surface at one end. However, when the body compressor is taken off from the upper arm and stored, the hook surface at the other end passes over the loop surface at one end and then contacts a front cover in the vicinity of one end if the other end of the band is excessively inserted in the metal clip. In this case, the front cover in the vicinity of one end rubs against the hook surface at the other end. Therefore, there is a problem that the front cover fluffs and thus spoils the beauty.

The present invention has been proposed to solve the problems of the conventional techniques described above. It is therefore an object of the present invention to provide a body compressor and a blood pressure monitor, wherein an overinsertion preventing member is provided to prevent the other end of a band from being excessively inserted in a metal clip when the body compressor is taken off from the upper arm and stored, in order to enable the improvement of operability and the prevention of fluffing of a front cover in the vicinity of one end due to friction.

### Means for Solving the Problems

In order to achieve the foregoing object, the present invention provides a body compressor including: a band in which an air bag is interposed between a front cover and a rear cover; and an annular metal clip which is attached to longitudinal one end of the band and into which the other end of the band is inserted, wherein an overinsertion preventing member which comes into contact with the metal clip is provided at a position a predetermined distance apart from one end of the band toward the other end thereof.

This ensures that the band is prevented from being disadvantageously reduced into a diameter smaller than the diameter corresponding to the predetermined distance. That is, prevention of the excessive reduction of the diameter of the band is ensured, so that when the body compressor is to be wound around the arm, there is no troublesome work of additionally expanding the diameter in a corresponding amount. This enables an improvement in operability.

In addition, in the present invention, the annular metal clip includes a completely annular metal clip, partly cut annular metal clip or partly detachable annular metal clip.

Preferably, the body compressor further includes fixing means for fixing the other end of the band folded via the metal clip, the fixing means being used as a hook-and-loop fastener, wherein a hook surface is provided on the other end side of the front cover, and a loop surface is provided from the hook surface to the vicinity of the one end.

Thus, the body compressor can be easily attached and removed. That is, when the body compressor is attached to, for example, the left upper arm, the body compressor can be easily attached and removed by pinching the other end with the right hand.

Preferably, the predetermined distance L₁ from one end of the band to the other end thereof is L₀/3<L₁<L₀/2, wherein L₀ is the entire length of the band.

Thus, when the body compressor is to be stored, the band is diametrically reduced until the overinsertion preventing member comes into contact with the metal clip, and if the band is folded via the metal clip, the hook surface of the hook-and-loop fastener is received on the loop surface. That is, the hook surface never passes over the loop surface and does not contact the front cover in the vicinity of one end, so that the hook surface never rubs against the front cover. This makes it possible to avoid the problem of the fluffing of the front cover that spoils the beauty.

Preferably, the overinsertion preventing member is provided at a position where this overinsertion preventing member contacts the metal clip when the diameter of the band is a measurable minimum diameter of the body compressor.

Thus, a user can easily know whether conditions for the measurable minimum diameter are satisfied. This makes it possible to enhance the added value.

In addition, the band is generally flexible, and is not capable of keeping its circular shape on its own. Therefore, in this specification, the diameter of the band means a virtual diameter on the assumption that the band keeps its circular shape.

Preferably, a joint of the air bag is protrusively provided on the one end side from the overinsertion preventing member.

This makes it possible to prevent the contact between the joint and the metal clip to disadvantageously cause damage to the joint or the root of the joint.

Preferably, the overinsertion preventing member is made of an elastic body shaped like an elongate rectangular plate, the elastic body being provided between the front cover and the air bag so that the longitudinal direction of the elastic body intersects with the longitudinal direction of the band.

Thus, when air is injected in the air bag, the sensation of a foreign object attributed to the overinsertion preventing member can be reduced. Moreover, the problem of impaired flexibility of the band can be avoided.

It should be understood that the overinsertion preventing member is not limited to the above-mentioned configuration. For example, the overinsertion preventing member may be provided with a hook surface on its rear surface, and removably attached to the loop surface of the hook-and-loop fastener. As a result, the position of the overinsertion preventing member can be freely adjusted. Therefore, for example, the overinsertion preventing member can be attached at a position suitable for the thickness of the upper arm of the user, and the winding amount (the degree of tightening by winding) can be stable.

Preferably, an anti-drop member which comes into contact with the metal clip is provided at the other end of the band.

Thus, even if the diameter of the band is excessively expanded, the other end of the band can be prevented from being disadvantageously dropping from the metal clip.

In order to achieve the foregoing object, a blood pressure monitor of the present invention uses the body compressor according to any one of claims 1 to 7.

Thus, the present invention is also effective in the blood pressure monitor, and can provide a blood pressure monitor having good operability and the like.

### BRIEF DESCRIPTION OF THE DRAWINGS

FIG. 1 is a schematic diagram of a body compressor according to one embodiment of the present invention, wherein (a) shows a front expansion plan and (b) shows a rear expansion plan;

FIG. 2 is a schematic diagram of the body compressor according to one embodiment of the present invention, wherein (a) shows a view in the arm direction when the diameter is enlarged and (b) shows a view in the arm direction when the diameter is reduced; and

FIG. 3 is a schematic diagram of the body compressor according to one embodiment of the present invention, wherein (a) shows a view in the arm direction when the body compressor is folded and (b) shows an enlarged view of a part A.

### BEST MODE FOR CARRYING OUT THE INVENTION

[One Embodiment of Body Compressor]

One embodiment of a body compressor of the present invention will be described below with reference to the drawings.

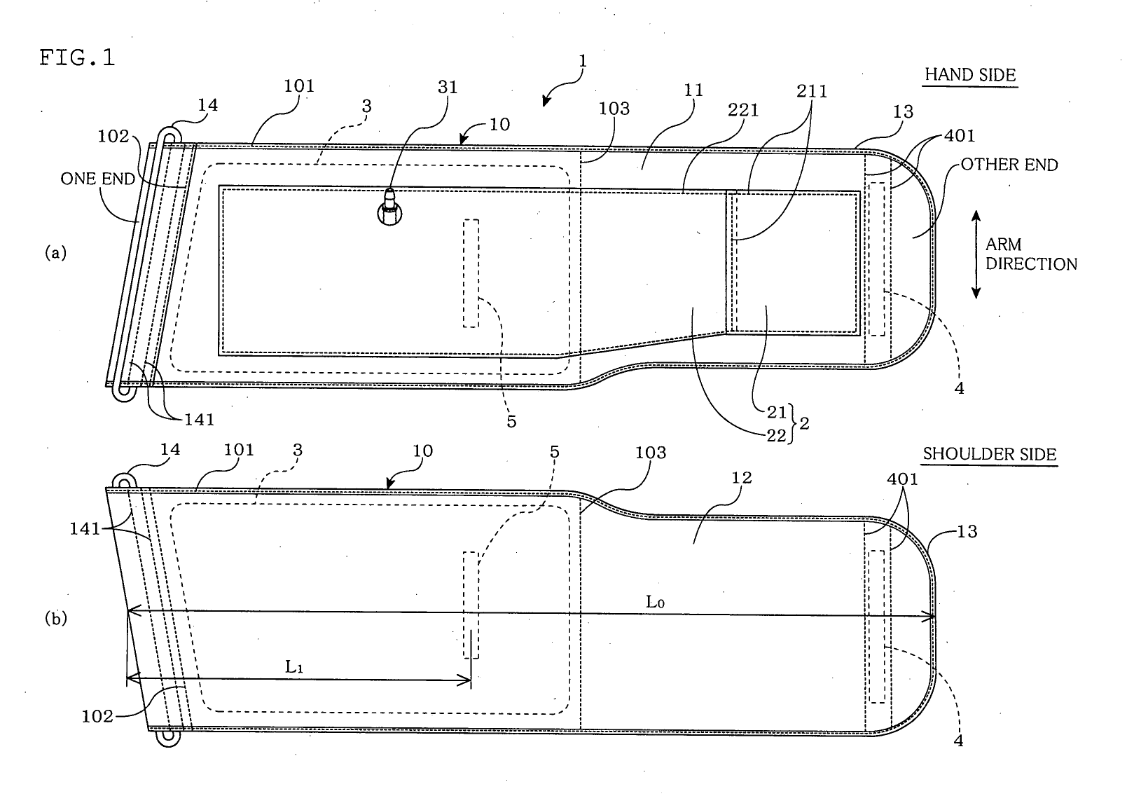

FIG. 1 is a schematic diagram of the body compressor according to one embodiment of the present invention, wherein (a) shows a front expansion plan and (b) shows a rear expansion plan.

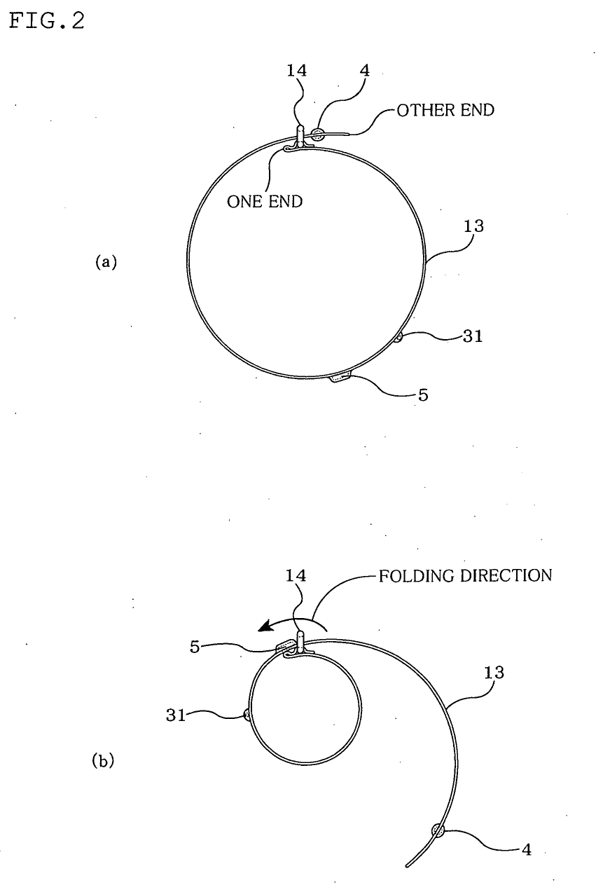

FIG. 2 is a schematic diagram of the body compressor according to one embodiment of the present invention, wherein (a) shows a view in the arm direction when the diameter is enlarged and (b) shows a view in the arm direction when the diameter is reduced.

In FIGS. 1 and 2, a body compressor 1 is used as a cuff for an automatic blood pressure monitor, and is wound around the upper arm of a user.

This body compressor 1 includes a band 10, a metal clip 14, a hook-and-loop fastener 2, an anti-drop member 4 and an overinsertion preventing member 5.

(Band)

The band 10 has an air bag 3, a front cover 11 and a rear cover 12. The air bag 3 is interposed between the front cover 11 and the rear cover 12.

The air bag 3 is substantially rectangular. The air bag 3 is affixed to the inside of the front cover 11 by, for example, a double-sided tape (not shown) along the longitudinal direction of the band 10. In the present embodiment, the front cover 11 and both longitudinal ends of the air bag 3 are affixed together by two double-sided tapes. When the double-sided tapes are thus used, the air bag 3 can be fixedly attached with ease. This enables a reduction in manufacturing costs. It should be understood that the present invention is not limited to this configuration. For example, sewing margins may be provided in the outer peripheral parts of both longitudinal ends of the air bag 3, and the sewing margins in the air bag 3 and the front cover 11 may be stitched together to fix the air bag 3 to the front cover 11.

Moreover, the air bag 3 has a protruding joint 31 on its outer surface to be coupled to a tube of the oscillometric electronic blood pressure monitor described above. This joint 31 is exposed outside through the front cover 11 and a loop surface 22.

Further, the air bag 3 is stored in a region ranging from substantially the longitudinal center of the band 10 to one end side.

Resin cloths, which are substantially rectangular, are generally used for the front cover 11 and the rear cover 12. The front cover 11 and the rear cover 12 are stitched together at their peripheral parts with a thread 101 via an edge cover 13 so as to interpose the air bag 3 therebetween. Substantially in the longitudinal center, the front cover 11 and the rear cover 12 are stitched together with a thread 103 along the arm direction.

Moreover, the front cover 11 and the rear cover 12 are smaller in width on the other end side of the thread 103 than on one end side thereof so as to easily fit to the shape of the arm.

(Metal Clip)

The metal clip 14 is an annular member made of a round bar. The other end of the band 10 is inserted into the metal clip 14. This metal clip 14 is attached to longitudinal one end of the band 10. That is, the pair of front and rear covers 11 and 12 are stitched together with a thread 141 so that the metal clip 14 does not move in a state interposed by the folded front cover 11 and the folded rear cover 12.

The metal clip 14 is obliquely attached at an angle of about 80° with the lower side (shoulder side) of the band 10. Thus, the body compressor 1 can be wound around even the muscular upper arm substantially in close contact with each other.

In addition, the ends of the folded front cover 11 and the folded rear cover 12 are stitched together with a thread 102.

(Hook-and-Loop Fastener)

In the present embodiment, the hook-and-loop fastener 2 is provided as fixing means. The hook-and-loop fastener 2 is inserted into the metal clip 14, and fixes the other end of the band 10 folded via the metal clip 14.

The hook-and-loop fastener 2 includes a hook surface 21 and the loop surface 22. The hook surface 21 is substantially square, and is stitched onto the surface of the front cover 11 on the other end side with a thread 211. The loop surface 22 is substantially rectangular, and is stitched onto the surface of the front cover 11 with a thread 221 from the hook surface 21 to the vicinity of one end.

Thus, the body compressor 1 can be easily attached and removed. That is, when the body compressor 1 is attached to, for example, the left upper arm, the body compressor 1 can be easily attached and removed by pinching the other end with the right hand.

While the hook-and-loop fastener 2 is used as the fixing means in the present embodiment, the present invention is not limited to this.

(Anti-Drop Member)

The anti-drop member 4 is a cylindrical member made of a resin. This anti-drop member 4 is provided at the other end of the band 10 so as to be interposed between the front cover 11 and the rear cover 12. In order to prevent the movement of the anti-drop member 4 in the present embodiment, the front cover 11 and the rear cover 12 on both sides of the anti-drop member 4 are stitched together with a thread 401.

The anti-drop member 4 is elastically deformed to lose its cylindrical shape and thereby inserted into the metal clip 14 (see FIG. 2(a)). However, the anti-drop member 4 after passed through the metal clip 14 is restored to the cylindrical shape. Thus, the other end comes into contact with the metal clip 14 even if the other end attempts to come out of the metal clip 14. This makes it possible to prevent the other end of the band 10 from disadvantageously dropping from the metal clip 14 even if the diameter of the band 10 is increased.

(Overinsertion Preventing Member)

The overinsertion preventing member 5 is a member to be in contact with the metal clip 14. The overinsertion preventing member 5 is provided at a position a predetermined distance L₁ apart from one end of the band 10 toward the other end thereof. This ensures that the band 10 can be surely prevented from being disadvantageously reduced to a diameter smaller than the diameter corresponding to the predetermined distance (see FIG. 2(b)). That is, the diameter of the band 10 can be prevented from being excessively reduced. As a result, there is no need for troublesome work of additionally expanding the diameter in a corresponding amount (amount of the excessive reduction of the diameter) when the body compressor 1 is wound around the arm. This enables an improvement in operability.

The overinsertion preventing member 5 in the present embodiment is an elastic body shaped like an elongate rectangular plate. The overinsertion preventing member 5 is provided between the front cover 11 and the air bag 3 so that the longitudinal direction of this elastic body intersects with the longitudinal direction of the band 10. That is, although not shown, the overinsertion preventing member 5 is stitched onto the front cover 11 with a thread. Thus, when air is injected in the air bag 3, the sensation of a foreign object attributed to the overinsertion preventing member 5 can be reduced. Moreover, the problem of impaired flexibility of the band 10 can be avoided.

Here, preferably, the predetermined distance L₁ from one end of the band 10 to the other end thereof is L₀/3<L₁<L₀/2, wherein L₀ is the entire length of the band 10 (the entire length in the center of the width direction (see FIG. 1(b))).

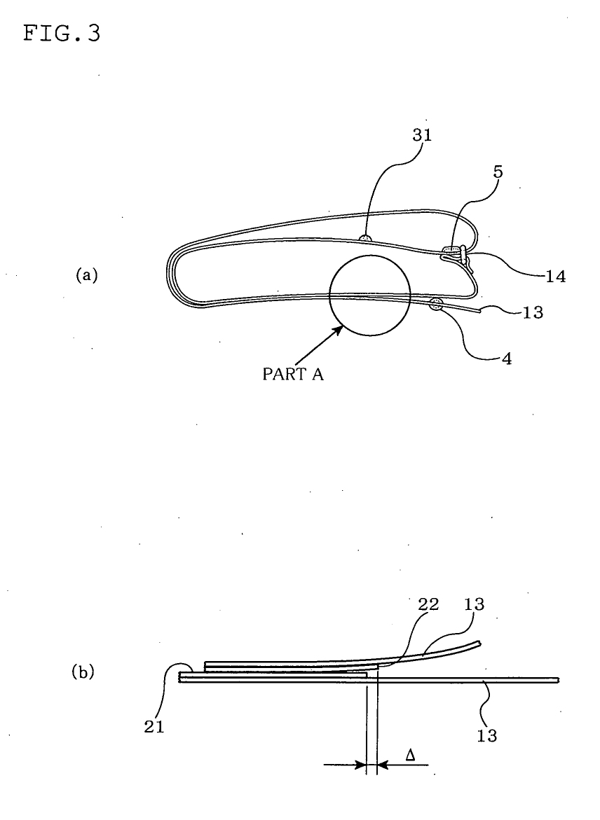

Thus, when the body compressor 1 is to be stored, the band 10 is diametrically reduced until the overinsertion preventing member 5 comes into contact with the metal clip 14, and if the band 10 is folded via the metal clip 14, the hook surface 21 of the hook-and-loop fastener 2 is received on the loop surface 22 (see FIG. 3). That is, the hook surface 21 never passes over the loop surface 22 and does not contact the front cover 11 in the vicinity of one end, so that the hook surface 21 never rubs against the front cover 11. This makes it possible to avoid the problem of the fluffing of the front cover 11 that spoils the beauty.

The overinsertion preventing member 5 may be provided at a position where this overinsertion preventing member 5 contacts the metal clip 14 when the diameter of the band 10 is a measurable minimum diameter of the body compressor 1. Thus, the user can easily know whether conditions for the measurable minimum diameter are satisfied. This makes it possible to enhance the added value.

Furthermore, the joint 31 of the air bag 3 in the present embodiment is provided protrusively toward the front surface through the front cover 11 and the loop surface 22. In this case, the joint 31 of the air bag 3 should be protrusively provided on the one end side from the overinsertion preventing member 5. As a result, the overinsertion preventing member 5 contacts the metal clip 14 before the joint 31 contacts the metal clip 14, thereby preventing excessive insertion. That is, the joint 31 does not contact the metal clip 14, thereby making it possible to ensure that the joint 31 or the root of the joint 31 is disadvantageously damaged.

Now, the operation and effects of the body compressor 1 having such a configuration are described.

First, the user to have his/her blood pressure measured inserts the arm into the body compressor 1 in which the band 10 is expanded at the maximum. Then, when the body compressor 1 has reached the upper arm, the other end is pulled. Thus, the band 10 is wound around the upper arm. Further, the band 10 is folded via the metal clip 14, and the hook surface 21 is joined to the loop surface 22. Here, as shown in FIG. 2(a), the anti-drop member 4 can maintain the maximum expansion of the band 10, so that the operability of the body compressor 1 improves.

Then, when the user finishes the blood pressure measurement, the other end of the band 10 is pulled, and the hook surface 21 comes away from the loop surface 22. Further, the body compressor 1 is taken off from the arm while the band 10 is being expanded.

Then, the user stores the body compressor 1. That is, as shown in FIG. 2(b), the band 10 is diametrically reduced until the overinsertion preventing member 5 comes into contact with the metal clip 14. Further, the other end of the band 10 is moved in a folding direction so that the hook surface 21 may be joined to the loop surface 22. In this state, four layers of the band 10 are stacked up as shown in FIG. 3(a), thereby saving storage space. Further, in the body compressor 1 in this state, the hook surface 21 of the hook-and-loop fastener 2 is received on the loop surface 22 so as to allow for a margin of a distance Δ, as shown in FIG. 3(b). That is, the hook surface 21 never passes over the loop surface 22 and does not contact the front cover 11 in the vicinity of one end. Thus, the hook surface 21 never rubs against the front cover 11. This makes it possible to avoid the problem of the fluffing of the front cover 11 that spoils the beauty.

Then, the user to have his/her blood pressure measured by use of the body compressor 1 in a stored state first pulls the other end of the band 10 starting from the state shown in FIG. 3(a), thereby separating the hook surface 21 from the loop surface 22. Further, the body compressor 1 is brought into the state shown in FIG. 2(b). Here, while the band 10 is diametrically reduced, the body compressor 1 has a predetermined diameter regulated by the overinsertion preventing member 5. As a result, the band 10 can be pinched with the hand and thus easily increased in diameter (see FIG. 2(a)).

Thus, according to the body compressor 1 in the present embodiment, the overinsertion preventing member 5 is provided such that the other end of the band 10 can be prevented from being excessively inserted into the metal clip 14. This enables a significant improvement in operability. Moreover, the body compressor 1 can be compactly stored with ease. When the body compressor 1 is to be stored, the hook surface 21 never rubs against the front cover 11 in the vicinity of one end. This makes it possible to effectively avoid the problem of the fluffing of the front cover 11 in the vicinity of one end that spoils the beauty.

In addition, the present invention is also advantageous to a blood pressure monitor used in the body compressor 1. The blood pressure monitor of the present invention has about the same effects as the body compressor 1. That is, it is possible to provide a blood pressure monitor having good operability.

The preferred embodiment of the body compressor and the blood pressure monitor according to the present invention has been illustrated above. However, it goes without saying that the body compressor and the blood pressure monitor according to the present invention are not exclusively limited to the embodiment described above, and various modifications can be made within the scope of the present invention.

For example, the body compressor 1 is not exclusively used in the oscillometric electronic blood pressure monitor. The body compressor 1 can also be applied to, for example, an automatic blood pressure monitor which measures a blood pressure on the basis of Korotkoff sounds.

Furthermore, the overinsertion preventing member 5 is not limited to the configuration described above. For example, although not shown, the overinsertion preventing member may be provided with a hook surface on its rear surface, and removably attached to the loop surface 22 of the hook-and-loop fastener 2 which is stitched onto the front cover 11. As a result, the position of the overinsertion preventing member can be freely adjusted. Therefore, for example, the overinsertion preventing member can be attached at a position suitable for the thickness of the upper arm of the user, and the winding amount (the degree of tightening by winding) can be stable.

### INDUSTRIAL APPLICABILITY

As described above, the body compressor of the present invention is not exclusively used in a blood pressure monitor cuff. For example, the present invention can also be effectively applied to a stanching body compressor which requires an accurate measurement of a pulse wave during pressure elevation.

1. A body compressor including: a band in which an air bag is interposed between a front cover and a rear cover; and an annular metal clip which is attached to longitudinal one end of the band and into which the other end of the band is inserted,
wherein an overinsertion preventing member, which comes into contact with the metal clip when the other end of the band is inserted into the annular metal clip and the diameter of the band is a measurable minimum diameter of the body compressor, is provided in or on the band.

2. The body compressor according to claim 1, which further includes fixing means for fixing the other end of the band folded via the metal clip, the fixing means being used as a hook-and-loop fastener, wherein a hook surface is provided on the other end side of the front cover, and a loop surface is provided from the hook surface to the vicinity of the one end.

3. The body compressor according to claim 2, wherein the predetermined distance L₁ from one end of the band to the overinsertion preventing member is L₀/3<L₁<L₀/2, wherein L₀ is the entire length of the band.

4. (canceled)

5. The body compressor according to claim 1, wherein a joint of the air bag is protrusively provided on the one end side from the overinsertion preventing member.

6. The body compressor according to claim 1, wherein the overinsertion preventing member is made of an elastic body shaped like an elongate rectangular plate, the elastic body being provided between the front cover and the air bag so that the longitudinal direction of the elastic body intersects with the longitudinal direction of the band.

7. The body compressor according to claim 1, wherein an anti-drop member which comes into contact with the metal clip is provided at the other end of the band.

8. A blood pressure monitor using the body compressor according to claim 1.
